(19) United States
(12) Patent Application Publication
(10) Pub. No.: US 2026/0043037 A1
(43) Pub. Date: Feb. 12, 2026

(54) EFFICIENT GENOTYPE-INDEPENDENT IN PLANTA TRANSFORMATION OF CEREALS

(71) Applicant: Purdue Research Foundation, West Lafayette, IN (US)

(72) Inventors: Mohsen Mohammadi, West Lafayette, IN (US); Cankui Zhang, West Lafayette, IN (US); Nirman Nepal, West Lafayette, IN (US)

(73) Assignee: Purdue Research Foundation, West Lafayette, IN (US)

(21) Appl. No.: 19/100,347

(22) PCT Filed: Aug. 1, 2023
(86) PCT No.: PCT/US2023/029230

§ 371 Date: Jan. 31, 2025

Related U.S. Application Data
(60) Provisional application No. 63/394,019, filed on Aug. 1, 2022.

(51) Int. Cl. C12N 15/82

(52) U.S. Cl. CPC C12N 15/8205

(57) ABSTRACT

Transgenic methods and constructs enable efficient genotype-independent in planta transformation of cercal crop species. A method of transforming cereals in planta includes co-incubating, post-anthesis, a cereal inflorescence of developing embryos with an inoculation medium comprising a transgene-modified Agrobacterium; collecting, upon the cereal inflorescence reaching maturity, seeds produced from at least a portion of the developing embryos; and selecting one or more transgenic seeds from among the collected seeds.

### CROSS-REFERENCE TO RELATED APPLICATIONS

This application claims priority to U.S. provisional patent application No. 63/394,019, filed Aug. 1, 2022.

### STATEMENT OF GOVERNMENT SUPPORT

This invention was made with government support under 58-5020-9-011 awarded by the United States Department of Agriculture. The government has certain rights in the invention.

### TECHNICAL FIELD

The present disclosure relates to genotype independent in planta transformation of wheat and other cereal crop plants.

### BACKGROUND

Genetic transformation in the hexaploid wheat genotype ‘Bobwhite’ was achieved in 1992 by Monsanto (Cheng et al., Plant Physiol 115:971-980 (1997)). High-velocity microprojectile bombardment was used to create an herbicide-resistant wheat variety (Vasil et al., Nat Biotech 10:667-674 (1992)). Using an immature seed as an explant, Agrobacterium-mediated tissue culture was used to transform the wheat variety ‘Fielder’ with a 40-90% transformation efficiency in Japan in 2015 (Ishida et al., Agrobacterium Protocols, Vol. 1, pp. 189-198, Wang, K., Ed. Springer (2015)). This method was used in six Australian wheat cultivars with 6-45% transformation efficiency (Richardson et al., Plant Cell Tiss Organ Cult 119:647-659 (2014)) and in sixteen Chinese commercial wheat cultivars with 2.9-22.7% transformation efficiency (Wang et al., Plant Biotechnol 15(5):614-623 (2017)). The most important issue with these tissue culture-based methods is their genotype dependency, which means only a handful of varieties can be transformed, even though thousands of wheat varieties, which differ in their agricultural importance, exist. For example, the two most commonly used wheat varieties, which have been successfully transformed, are ‘Fielder’ and ‘Bobwhite’ but neither one is currently commercially cultivated anywhere in the world. In addition, the very low transformation efficiency, if it works on rare varieties other than ‘Fielder’, and the amount of time to generate T₀ plants, i.e., ˜1 year, are bottlenecks for the application of methods such as genome editing.

Recent studies have shown that the transformation efficiency and genotype dependency of wheat transformation can be overcome by overexpressing WUSCHEL family transcription factor (TaWOX5) using callus culture (Wang et al., Nat Plants 8:110-117 (2022)). This method, which is based on overexpressing other morphogenic genes, i.e., booster genes, can improve transformation efficiency but it is cumbersome, time-consuming, requires ample resources, and comes with the adverse phenotypic effects of booster gene overexpression in the transgenic wheats (Wang et al. (2022), supra).

In planta transformation, on the other hand, which does not require the need for tissue culture, may overcome the long procedural duration for transformation. To date, few studies have shown in planta wheat transformation. Earlier researchers have sprayed plant inflorescence structures (Zale et al., Plant Cell Rep 28:903-913 (2009)) or inoculated embryos from detached spikes, followed by tissue culture to rescue the immature embryo with a low transformation efficiency of ˜5% (see, for example, Int'l Pat App Pub No. WO 00/63398; and Risacher et al., Methods Mol Biol 478:115-124 (2009)). When researchers sprayed floral parts with Agrobacterium, the transformation efficiency was 0.12 and 0.23% in two varieties, i.e., HD 2967 and Bobwhite, but transformation was unsuccessful in the variety HD3086 (Singh and Kumar, Plant Biochem Biotechnol 31:206-212 (2022)). This indicated that this method is not only inefficient but genotype-dependent.

Dan et al. (U.S. Pat. Nos. 10,253,322 and 9,353,377) discloses a method for transformation of varieties of wheat, which reportedly is genotype-independent. The method, however, involves culture of mesocotyl meristem explants that contain multiple primary meristems.

Hence, there is a long-felt, unmet need of an efficient, genotype-independent method of transforming wheat and other cereal crop species that is independent of tissue culture. In view of the foregoing, it is an object of the present disclosure to provide such a method. This and other objects and advantages, as well as inventive features, will be apparent from the detailed description provided herein.

### SUMMARY

A method of transforming a wheat or other cereal in planta is provided. The method comprises: (i) co-incubating, post-anthesis, a cereal inflorescence of developing embryos with an inoculation medium, wherein the inoculation medium comprises a transgene-modified Agrobacterium; (ii) collecting, upon the cereal inflorescence reaching maturity, seeds produced from at least a portion of the developing embryos; and (iii) selecting one or more transgenic seeds from among the collected seeds. The method may further comprise exposing at least a portion of the developing embryos prior to co-incubation by trimming awns, removing outer glumes, and plucking out middle florets of each respective embryo. The exposed embryos may also be wounded prior to co-incubation. The exposed embryos may be wounded at about 7 days to about 24 days after anthesis.

The cereal inflorescence may be co-incubated with the inoculation medium comprising a transgene-modified Agrobacterium by immersing the inflorescence in the inoculation medium. The cereal inflorescence may be immersed in the inoculation medium immediately after the exposed, developing embryos are wounded. The cereal inflorescence may be co-incubated with the inoculation medium for about 10 hours to about 72 hours and then covered until maturity. After co-incubation, the cereal inflorescence may be covered with a paper bag. The cereal inflorescence may be exposed to light at about 90 to about 1,500 μmolm⁻²s⁻¹ for up to about 16 hours daily. The cereal inflorescence may be co-incubated with the inoculation medium at a temperature of about 25±5° C. The inoculation medium may be shaken gently once every 6 hours to about every 12 hours.

Alternatively, the cereal inflorescence is co-incubated with the inoculation medium under vacuum infiltration. The vacuum infiltration may be at about 55 PSI. The vacuum infiltration may last from about 10 minutes to about 60 minutes. In one example, the vacuum infiltration lasts about 30 minutes. After vacuum infiltration, the cereal inflorescence may be maintained at a temperature of about 20±5° C. The cereal inflorescence may be exposed to light at about 90 to about 1,500 μmolm⁻²s⁻¹ for up to about 16 hours daily.

The Agrobacterium may be a strain selected from the group consisting of AGL1, EHA105, GV1301, At503, LBA4404, EHA101, and C58C1. In one example, the Agrobacterium is the strain AGL1. The transgene may be in a vector selected from pCAMBIA1305.1, PIP2-GUS-Bar, or a combination thereof.

The cereal may be selected from Triticum (wheat), Sorghum (sorghum), Oryza (rice), Avena (oats), Zea (corn), Hordeum (barley), and Secale (rye). In one example, the cereal is a Triticum (wheat) variety. The Triticum (wheat) variety may be selected from AG0762, Apogee, Bobwhite, Cadenza, Canvas, Central Red, Chinese spring, Cranbrook, Fielder, Gallagher, Gilat, Glenlea, Halbert, IN0316, Inia 66, Ke-Qun, Kronos, Lassik, Line 10, Line 28, Line 43, NacozariF76, Opata, Patwin, Red Fife, Ruby, S-24, Seri-82, Sky dance, Smith's Gold, Sonora64, T-13, T-27, T-28, T-38, Ulen, Veery 19, Verde, and Yecora-Rojo. In one example, the cereal is a Hordeum (barley) variety. The Hordeum (barley) variety may be selected from Rasmusson, Quest, and Robust. In one example, the cereal is an Avena (oats) variety selected from INO9201 and Excel. In one example, the cereal is a Sorghum (sorghum) variety. The Sorghum (sorghum) variety may be selected from RTX423 and TX623.30.

The method may further comprise (iv) growing a plant from each of one or more of the transgenic seeds of (iii); (v) collecting seeds from the plants of (iv); and (vi) selecting for transgenic seeds among the collected seeds of (v). A transgenic plant obtained in accordance with the method is provided. A cell, tissue, or organ obtained from the transgenic plant is also provided. Further provided is a transgenic seed obtained from the transgenic plant. Still further provided is a T₂ plant obtained from the transgenic seed obtained from the transgenic plant.

### BRIEF DESCRIPTION OF THE FIGURES

FIG. 1A depicts a spike and spikelet during maturity, indicating developed seeds and outer glumes.

FIG. 1B depicts a seed of an Apogee cultivar with a wounded, exposed embryo.

FIG. 1C depicts a wild-type mature seed with a wounded, exposed embryo.

FIG. 1D depicts evidence of wounding in mature seed.

FIG. 2A depicts preprocessed wheat spikes co-incubated for 48 hours with Agrobacterium in inoculation media.

FIG. 2B depicts preprocessed wheat spikes vacuum infiltrated with Agrobacterium in inoculation media.

FIG. 2C depicts spikes covered by paper bags after co-incubation or vacuum infiltration.

FIG. 3A depicts selection of T₀ transgenic wheat accession (Opata-85) in antibiotic selection media 200 mg/L hygromycin and 160 mg/L timentin.

FIG. 3B depicts GUS staining of wheat seedlings.

FIG. 3C depicts PCR amplification of Hyg resistance gene in antibiotic resistant seedlings.

FIG. 3D is a bar graph comparing transformation efficiency on twenty-eight wheat accessions and two T. monococcum accessions, where error bars are based on three plants as each experimental unit.

FIG. 3E depicts a southern blot analysis of T₀ transformants, where WT=wild-type, T=Transformed, PCR product size=395 bp, PC=Plasmid positive control, NTC=Non-template control.

FIG. 4A depicts selection of T₁ seeds of Yecora-Rojo wheat variety in antibiotic selection media (200 mg/l hygromycin).

FIG. 4B depicts PCR amplification of Hyg resistance gene, PCR amplicon size=395 bp, PC=plasmid control, WT=wild-type.

FIG. 4C depicts GUS staining of root specimens of T₁ plants.

FIG. 4D depicts GUS staining of T₁ Yecoro rojo seedling.

FIG. 4E depicts GUS staining of T₁ Opata seedling.

FIG. 5A depicts oat (INO9201) accession transformed.

FIG. 5B depicts PCR amplification of GUS gene in transformed oat accession, PCR amplicon size=195 bp.

FIG. 5C depicts GUS staining of oat T₀ transformant leaves.

FIG. 5D depicts barley accession transformed.

FIG. 5E depicts PCR amplification of GUS in barley accession, PCR amplicon size=195 bp.

FIG. 5F depicts GUS staining of barley T₀ transformant leaves.

FIG. 6A depicts putative sorghum T₀ plants.

FIG. 6B depicts selection of sorghum putative T₀ in antibiotic selection media using 200 mg/l hygromycin.

FIG. 6C depicts PCR amplification of GUS gene, PCR amplicon size=195 bp.

FIG. 6D depicts screening of sorghum transformants with Basta painting; Basta concentration 2% v/v, and pictures were obtained one week after Basta painting.

### DETAILED DESCRIPTION

Methods of transforming a cereal crop species (hereafter “cereal”) in planta are provided, as well as transfected seeds generated in accordance with such methods, transgenic plants grown from transfected seeds, and successive progeny.

The present disclosure is predicated, at least in part, on the surprising and unexpected discovery of a highly efficient, genotype-independent method of transforming Triticum (wheat). The transformation is independent of tissue culture, reproducible, and not accompanied by any observable adverse effects on the plants. The method is also applicable to other cereals, such as Sorghum (sorghum), Oryza (rice), Avena (oats), Zea (corn), Hordeum (barley), and Secale (rye), for example.

In view of the foregoing, a method of transforming cereals in planta is provided. The method will be described initially for transforming wheat in planta and is applicable to other cereals with the variations further described herein. As used herein, “transforming” means to change in a heritable manner the characteristics of a host cell in response to DNA foreign to that cell. The method produces transgenic seeds that may be used to further produce transgenic plants, including successive generations of transgenic plants.

Transformation here includes methods to obtain transgenic, xenogenic, intragenic and cisgenic transformation and the term “transgenic,” herein, describes a seed or plant transformed through transgenesis, xenogenesis, intragenesis and cisgenesis. The term “transgene” further refers to any polynucleotide sequence introduced into the genome of a T₀ generation plant cell through genetic engineering. Polynucleotide sequences contemplated herein may be endogenous or heterologous and may encompass a wide variety of genetic materials—e.g., gDNA, synthetic DNA, mRNA, non-coding RNA, and small interfering RNA (siRNA), micro-RNA (miRNA), complimentary nucleotide sequences, coding and non-coding sequences, polynucleotide conjugates and analogues, synthetic and recombinant polynucleotides, and amplicons or clones of sample genetic material. A transgene herein can cause the expression through of one or more cellular products, including, e.g., transcription and post-transcription polynucleotides, polypeptides, and proteins. Exemplary transgenes may provide the host cell, or plants regenerated therefrom, with a novel phenotype relative to the corresponding non-transformed wild-type allele, cell and/or plant. Transgenes may be directly introduced into a plant by genetic transformation, or may be inherited from a plant of any previous generation which was transformed with the transfected polynucleotide segment.

Transgenes herein may be operably linked to a promoter in a transgene-promoter construct selected to yield a given species-specific expression pattern. Transgenes may additionally or alternatively be incorporated into a carrier or vector construct, including recombinant nucleic acid sequence vectors, e.g., in plasmid or replicon forms, which may further comprise a selectable marker gene, reporter gene, enhancer element, poly (A) sequence, ribosomal binding sequence, and/or transit peptide DNA sequence.

Transgenes contemplated herein may be employed with a variety of transformation strategies. For example, methods and constructs herein may be employed in connection with various genome editing techniques, including gene editing via, e.g., exogenously supplied repair templates for targeted double-strand breaks generated using sequence-specific nucleases. Such nucleases may include, e.g., meganucleases, zinc-finger nucleases (ZFNs), transcription activator-like effector nucleases (TALENs), and the clustered, regularly interspaced, short palindromic repeat (CRISPR)-associated protein (Cas) systems. Gene editing herein may modify plant genomes in numerous ways, ranging from several nucleotide substitutions to the targeted deletion of megabases of DNA, and may include whole gene insertion or excision (knock out) of trait genes of interest, editing of promoter or regulatory sequences, or modifications of genome region of interests for mutant-type expression based on small variants, e.g., single nucleotide polymorphisms (SNPs), or large variants, e.g., structural variants (SVs), and may involve either inducing or correcting deletions, insertions, inversions, tandem repeats and/or translocations within such regions. As another example, methods and constructs herein may be deployed in connection with mediation of post-transcriptional expression of target genes. Transgenes may be selected or engineered to knock down expressions via, e.g., RNA interference (RNAi). In certain examples, a transgene may comprise a short interfering RNA (siRNA), a micro-RNA (miRNA), or cDNA encoding an siRNA or miRNA. Transgenes may be selected or engineered to promote expression or overexpression. In certain examples, the transgene encodes an RNA silencing suppressor protein for suppressing natural cellular RNAi processes. Transgenes may also be selected or engineered to modulate epigenomic functioning, including, e.g., the up-regulation or down-regulation of various other cellular components involved in transcriptomic or proteomic processes.

Methods and constructs herein may be implemented in a variety of contexts. For example, in certain embodiments, methods and constructs herein may be used for expression of a desired phenotypic, metabolic, transcriptomic, and proteomics outcome, in either a commercial or laboratory context. In other embodiments, methods and constructs herein may be implemented in an experimental context, e.g., targeted mutagenesis, deletion analysis, and gain of function experiments.

Methods of transforming a wheat or other cereal in planta is provided. In one example the method comprises: (i) co-incubating, post-anthesis, a cereal inflorescence of developing embryos with an inoculation medium, wherein the inoculation medium comprises a transgene-modified Agrobacterium; (ii) collecting, upon the cereal inflorescence reaching maturity, seeds produced from at least a portion of the developing embryos; and (iii) selecting one or more transgenic seeds from among the collected seeds. The one or more transgenic seeds may be selected using selectable marker and reporter genes, for example, herbicide resistance gene, antibiotic resistance gene, GUS expression, fluorescence (GFP/YFP/RFP) or luminescence. While (i) can be carried out at any point post-anthesis, transformation efficiency can be maximized within about one week to about four weeks (e.g., about one week to four weeks, one week to about four weeks, or one week to four weeks), such as within about two weeks to about four weeks (e.g., about two weeks to four weeks, two weeks to about four weeks, or two weeks to four weeks), of anthesis. The cereal inflorescence in (i) is intact, i.e., not detached from the remainder of the plant. The seeds are produced from the maturing embryos. Transgenic seeds are produced from embryos that have been transformed in (i).

Maturity as used herein refers to the terminal growth stage of the cereal inflorescence, as marked by senescence of plant foliar tissues as seen in the greenhouse and fields of cereal crops, and which can also be determined, e.g., by grain moisture content, level of kernel hardness due to terminal growth desiccation, or dry matter accumulation.

Transgenes herein may be delivered through suitable phytopathogenic-based models other than Agrobacterium, including, for example, transfer DNA (T-DNA) binary vector systems, engineered yeasts, artificial chromosomes, plasmids, cosmids, phages, viruses, viroids, capsids, and transposons. Delivery may also be affected via nano-encapsulation or other nano-construct, circularized recombinant polynucleotides, or free polynucleotides in suspension or solution.

In another embodiment, transformation may be carried out through direct injection of a solution carrying a transgene into an exposed developing embryo of an intact inflorescence. In certain examples, a transgene of the solution may be carried in a phytopathogenic vector construct such as Agrobacterium described herein. In certain other examples, the transgene is “free” in solution, where it may be carried, e.g., in a naked plasmid or linear DNA construct. In one such example, the method comprises (i) post-anthesis, injecting into one or more exposed, wounded developing embryos of an inflorescence with solution containing transgene carried in a linear DNA construct that includes, e.g., a promotor, a read frame and a terminator; (ii) when the cereal inflorescence reaches a point of physiological maturity, collecting seeds; and (iii) selecting for transgenic seeds among the collected seeds using selectable marker and reporter genes for example, herbicide resistance gene, antibiotic resistance gene, GUS expression, fluorescence (GFP/YFP/RFP) or luminescence.

Methods herein may further comprise exposing at least a portion of the developing embryos prior to co-incubation by trimming awns, removing outer glumes, and plucking out middle florets of each respective embryo.

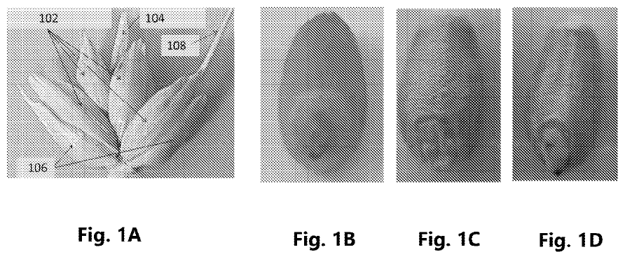

The method may further comprise, prior to co-incubating the cereal inflorescence with the inoculation medium, pre-processing the cereal inflorescence by removing external parts that interfere with access to the embryos. For example, with reference to FIG. 1A, the method may further comprise exposing at least a portion of the developing embryos (within florets 102) prior to co-incubation, e.g., by trimming awns 108, removing outer glumes 106, and plucking out middle florets (from among florets 102) of the developing embryos. Infertile florets 104 may also be plucked out. The exposed, developing embryos may also be wounded prior to co-incubation. The exposed, developing embryos can be wounded at about 7 days to about 24 days (e.g., about 7 days to 24 days, 7 days to about 24 days, 7 days to 24 days, about 18 days to about 24 days, about 18 days to 24 days, 18 days to about 24 days, or 18 days to 24 days, depending on variety) after anthesis. Wounding may be accomplished through various techniques including, for example, via incision, scraping, particle bombardment, or ultrasound. Different cereal varieties and different genotypes may have different development times for embryos, and those development times can be impacted by environmental conditions, such as temperature condition post-anthesis. Different cereal varieties and different genotypes can also vary in kernel, shape, and target area of incision in the kernel and embryo size. Hence, the best time for carrying out the wounding procedure vary with genotype and current environmental conditions. Examples of exposed, wounded developing wheat embryos are shown in FIG. 1B for the Apogee cultivar and in FIG. 1C for the wild type seed. FIG. 1D shows evidence of wounding in a mature seed.

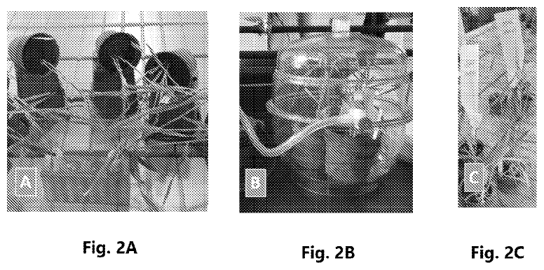

The cereal inflorescence can be co-incubated with the inoculation medium comprising Agrobacterium by immersing the inflorescence in the inoculation medium, i.e., submerging the cereal inflorescence into the inoculation medium while the cereal inflorescence is attached to the maternal plant. An example of preprocessed wheat spikes co-incubated for 48 hours with Agrobacterium in inoculation media is shown in FIG. 2A. The cereal inflorescence can be completely immersed in the inoculation medium immediately after the exposed, developing embryos are wounded. Without being bound by theory, it is believed that immersing the inflorescence at this point minimizes or prevents desiccation of the embryos and is the point when the plant is most susceptible to Agrobacterial infection. The cereal inflorescence can be co-incubated with the inoculation medium for about 10 hours to about 72 hours (e.g., about 10 hours to 72 hours, 10 hours to about 72 hours, 10 hours to 72 hours, about 24 hours to about 48 hours, about 24 hours to 48 hours, 24 hours to about 48 hours, or 24 hours to 48 hours) and then covered until maturity. The cereal inflorescence can be covered with a paper bag as shown in FIG. 2C. The cereal inflorescence can be co-incubated with the inoculation medium at a temperature of about 25±5° C. (e.g., about 25±2° C.). The cereal inflorescence, while intact on the mother plant, can be exposed to light at about 90 to about 1,500 μmolm⁻²s⁻¹ (e.g., about 1,000 μmolm⁻²s⁻¹) for up to about 16 hours daily (depending on variety). The inoculation medium can be occasionally shaken gently (e.g., from about every 6 hours to about every 12 hours).

Alternatively, the cereal inflorescence can be co-incubated with the inoculation medium comprising Agrobacterium under vacuum infiltration and then covered until maturity. Infiltration conditions will depend, at least in part, on the particular instrument used for vacuum infiltration as indicated in the manufacturer's guidelines. Infiltration conditions will also depend on the plant being infiltrated. Exemplary instrument infiltration conditions are shown in FIG. 2B. Wheat can be vacuum infiltrated at 55 PSI, for example. The vacuum infiltration can last from about 10 minutes to about 60 minutes, such as about 30 minutes. The cereal inflorescence can be covered with a paper bag. After vacuum infiltration, the cereal inflorescence can be maintained at a temperature of about 20±5° C. The cereal inflorescence can be exposed to light at about 90 to about 1,500 μmolm⁻²s⁻¹ for up to about 16 hours daily (depending on variety).

The Agrobacterium can be, for example, any gram-negative, rod-shaped phytopathogenic bacterium within the Agrobacterium species, including any suitable strain thereof. The Agrobacterium strain can be selected from the group consisting of Agrobacterium tumefaciens strains, Agrobacterium rhizogenes strains, agropine-type Agrobacterium strains, octopine-type Agrobacterium strains, nopaline-type Agrobacterium strains, strain AGL1, strain EHA105, strain At503, strain EHA101, strain LBA4404, strain C58C1 and strain GV1301. The AGL1 strain can have a higher transformation efficiency compared to other binary vectors. In certain examples, the Agrobacterium is a strain selected from the group consisting of AGL1, EHA105, GV1301, At503, LBA4404, EHA101, and C58C1. In one example, the Agrobacterium is the strain AGL1. The transgene may be in a vector selected, e.g., from pCAMBIA1305.1, PIP2-GUS-Bar, or a combination thereof.

Following co-incubation or injection of the developing embryo, the cereal inflorescence continues aging to a point of physiological maturity, and the seeds are then collected. This post-injection period for recovery of the plant and production of transgenic seeds may occur by allowing the seeds to grow naturally on the mother plant until ripening, or by collecting the embryos shortly after co-incubation or injection and matured using accelerated generation advancement techniques, or speed breeding techniques, or using embryo rescue techniques.

Any conditions suitable for growth and development of plants, such as cereals, in particular wheat, can be used. Exemplary conditions are provided in the Examples. The usual temperature for wheat is about 25° C. Some important cereals, such as corn, sorghum, and rice, need warmer temperatures for normal growth. Other cereals, such as wheat, rye, barley, and oat need cooler temperatures for normal growth.

The inoculation medium can include any suitable medium as is known in the art, such as Luria broth (LB), a sugar, such as sucrose (e.g., from about 2% to about 5%, such as 5%), and a nonionic surfactant, such as, but not limited to, a trisiloxane surfactant, e.g., Silwet L-77®, which is a mixture of about 84% polyalkyleneoxide modified heptamethyltrisiloxane and about 16% allyloxypolyethyleneglycol methyl ether (e.g., from greater than 0% to about 0.05%, such as about 0.02% or 0.02%). Other examples of sugars, which may be used in place of or in addition to sucrose, include but are not limited to glucose (dextrose), fructose, galactose, xylose, and ribose.

The selection of genes and promoters, as well as selectable markers, for Agrobacterium-mediated plant transformation is within the ordinary skill in the art. The ordinarily skilled artisan can select genes, which can impart desirable traits, e.g., agronomic traits, to the wheat. Likewise, methods of screening for transformants are also known in the art.

The method can be used to transform wheat independent of genotype. There are thousands of varieties of wheat world-wide. Examples of wheat varieties include, but are not limited to, 1863, 2137, 2145, 2174, Above, AG0762, AG Gallant, Alliance, Akron, Ankor, Antero, AP502CL, AP503CL2, Apogee, Art, Aspen, Avalanche, Avery, Baker's White, Betty, Bentley, Big Max, Bill, Bob Dole, Bobwhite, Bond CL, Brawl CL+, OK Bullet, Burchett, Byrd, Cadenza, Canvas, Centerfield, Central Red, Chinese spring, Cisco, Clara CL, Coronado, Cossack, Cougar, Cranbrook, Culver, Custer, Cutter, Danby, Deliver, Denali, Doans, Dominator, Doublestop CL+, Dumas, Duster, Eagle, Endurance, Enhancer, Everest, Fannin, Fielder, Freeman, Fuller, Gallagher, Gilat, Glenlea, Guymon, Halbert, Hallam, Halt, Harry, Hatcher, Hawken, Iba, Ike, Infinity CL, IN0316, Inia 66, Intrada, Jagalene, Jagger, Joe, KanMark, Karl/Kar192, Kcota, Kc-Qun, Kojak, Kronos, Langin, Larned, Larry, Lassik, LCS Chrome, LCS Link, LCS Mint, LCS Pistol, LCS Wizard, Line 10, Line 28, Line 43, Lockett, Lonerider, Longhorn, Millennium, NacozariF76, Neosho, Niobrara, NuDakota, NuFrontier, NuGrain, NuHills, Nullorizon, Nuplains, Ogallala, Oakley CL, Ok101, Ok102, Okfield, Onaga, Opata, Overland, Overley, Patwin, Platte, Postrock, Prairie Red, Prairie White, Protection, Prowers/Prowers 99, Red Fife, Ripper, Robidoux, RonL, Ruby, Ruby Lee, Ruth, S-24, Santa Fe, Seri-82, Shocker, Sky dance, Smith's Gold, Smoky Hill, Sonora64, Spirit Rider, Stardust, Stanton, Sturdy 2K, SY 517CL2, Achieve CL2, Benefit, Flint, Grit, Monument, Rugged, Sunrise and Wolf; T-13, T-27, T-28, T-38, T81, T81SV, T812, T83, T113G, T118, T129, T136, T140, T158, T193; TAM 105, 107, 110, 111, 112, 113, 114, 200, 202, 203, 204, 301, 302, 304 and 400; Tarkio, Thunderbolt, Ulen, Veery 19, Verde, Vista, Wahoo, WB4269, WB4303, WB4458, WB4515, WB4721, WB-Cedar, WB-Grainfield, Weathermaster 135, Wesley, Windstar, Winterhawk, Yecora-Rojo, Yuma, Yumar, and Zenda.

In certain example embodiments, the cereal is selected from Triticum (wheat), Sorghum (sorghum), Oryza (rice), Avena (oats), Zea (corn), Hordeum (barley), and Secale (rye). In one example, the cereal is a Triticum (wheat) variety. The Triticum (wheat) variety may be selected from AG0762, Apogee, Bobwhite, Cadenza, Canvas, Central Red, Chinese spring, Cranbrook, Fielder, Gallagher, Gilat, Glenlea, Halbert, IN0316, Inia 66, Ke-Qun, Kronos, Lassik, Line 10, Line 28, Line 43, NacozariF76, Opata, Patwin, Red Fife, Ruby, S-24, Seri-82, Sky dance, Smith's Gold, Sonora64, T-13, T-27, T-28, T-38, Ulen, Veery 19, Verde, and Yecora-Rojo. In one example, the cereal is a Hordeum (barley) variety. In one example, the Hordeum (barley) variety is selected from Rasmusson, Quest, and Robust. In another example, the cereal is an Avena (oats) variety selected from INO9201 and Excel. In yet another example, the cereal is a Sorghum (sorghum) variety selected from RTX423 and TX623.30.

The method, above, may further comprise growing a plant from each of one or more of the transgenic seeds of (iii); (v) collecting seeds from the plants of (iv); and (vi) selecting for transgenic seeds among the collected seeds of (v). A transgenic plant obtained in accordance with the method is provided. For example, transgenic wheat plants, transgenic oat plants, transgenic barley plants, and transgenic sorghum plants obtained in accordance with the above method are provided. Moreover, a cell, tissue, or organ obtained from a transgenic plant obtained in accordance with the method is also provided. Further provided is a transgenic seed obtained from a transgenic plant herein. Still further provided is a T₂ transgenic plant obtained from the transgenic seed obtained from the transgenic plant.

Although specific varieties of wheat, sorghum, rice, oats, corn, barley, and rye are referenced herein, it is expected that the present method may be used for other cereal crop varieties, including, but not limited to, heirloom wheat varieties, modern wheat varieties, and future wheat and other cereal varieties.

### EXAMPLES

The following examples serve to illustrate the present disclosure. The examples are not intended to limit the scope of the claimed invention in any way.

### Example 1

### Plant Growth, Pre-Processing, Wounding, and Inoculation.

Plants were grown in the greenhouse in a sandy soil mix (⅓ topsoil, ⅓ sand and ⅓ compost) at 25±5° C. under a photoperiod, which was best for the variety, e.g., such as a 16-hour photoperiod, at a level of approximately 1,000 μmolm⁻²s⁻¹ light intensity, unless otherwise indicated. Two to four weeks (i.e., around 18-25 days, depending on the variety) after anthesis, intact (and not detached) wheat florets with developing embryos were transformed.

Pre-processing included trimming of removal of glumes, awns, and unwanted florets using stainless steel inox straight cuticle scissor (3 swords, Germany) and pointed tip tweezers (Bardeau Essentials, US). It is recommended, though not required, that the middle spikes and spikes at the top of the spikelet be removed for easier access to the seeds/embryos without disturbing other seeds. Pre-processing of intact embryos for co-incubation should be performed carefully so the developing kernels do not fall off the spikelets.

Wounding involved making multiple incisions in exposed embryo tissue after anthesis using pointed tip tweezers (Bardeau Essentials, US).

Inoculation involved co-incubating entire spikes or inflorescence, i.e., dipping inflorescence in the inoculation medium, with Agrobacterium immediately after wounding to prevent desiccation of embryos.

Two different vectors were used. The first one was pCAMBIA1305.1, which consists of a hygromycin selectable marker with GUS-cat intron gene under the control of the CaMV35S promoter. The second one was pBISN1 in which the GUS gene is under the control of the synthetic Pnos promoter. Three Agrobacterium strains AGL1, EHA105, and GV1301 were used to find an appropriate Agrobacterium strain for transformation.

Agrobacterium tumefaciens harboring pCAMBIA1305.1 was cultured in LB containing kanamycin (50 mg/l) and incubated overnight at 28° C. with shaking at 225 rpm. When the Agrobacterium growth reached an OD₆₀₀ₙₘ of 0.8 or higher, the cultures were centrifuged at 3000×g for 10 minutes. The cell pellets were suspended in 5% sucrose to maintain the final OD₆₀₀ₙₘ of 0.8 to prepare inoculation media. Prior to co-cultivation, Silwet L-77 0.02% was added to the inoculation media, which was then used for transformation.

Two treatments were applied for inoculation. Both were successful but differed in transformation efficiency.

In treatment I, preprocessed wheat spikes were co-incubated with inoculation media for 24 or 48 hours at 25±2° C. in the greenhouse (FIG. 2A), and then were covered in paper bags until maturity (FIG. 2C) and placed in greenhouse at 25±5° C. under 16/8 hours daylight condition and ˜1000 μmolm⁻²s⁻¹ light intensity. Agrobacterium tends to settle (precipitate) at the bottom of the container so occasional (e.g., every 12 hours), gentle shaking of inoculum media was required.

In treatment II, spikes were co-incubated with Agrobacterium inoculation media under vacuum infiltration at 55 PSI for various lengths of time treatments i.e., 10 min, 30 min, and 60 min (FIG. 2B). After vacuum infiltration, spikes were wrapped in paper bags until physiological maturity (FIG. 2C) in the greenhouse 25±5° C. or a laboratory grow-shelf at 20±5° C., with 90-100 μmol/m²/s¹ light intensity and 8 hours photoperiod.

### Example 2

### Transgenic Screening and Estimating Transformation Efficiency.

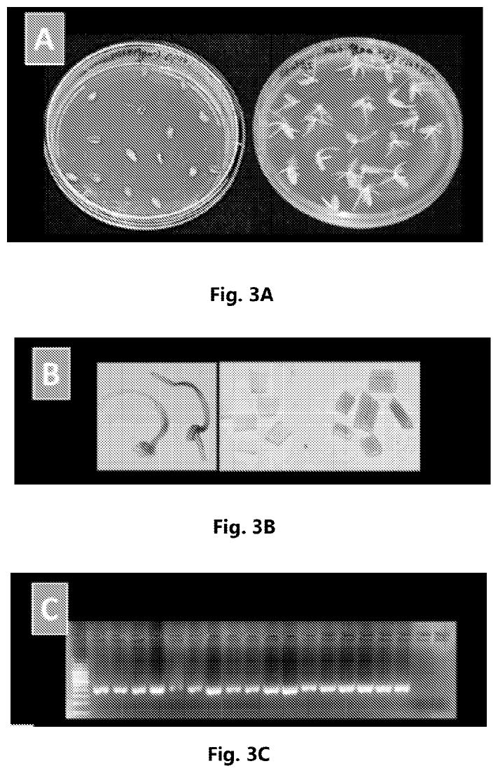

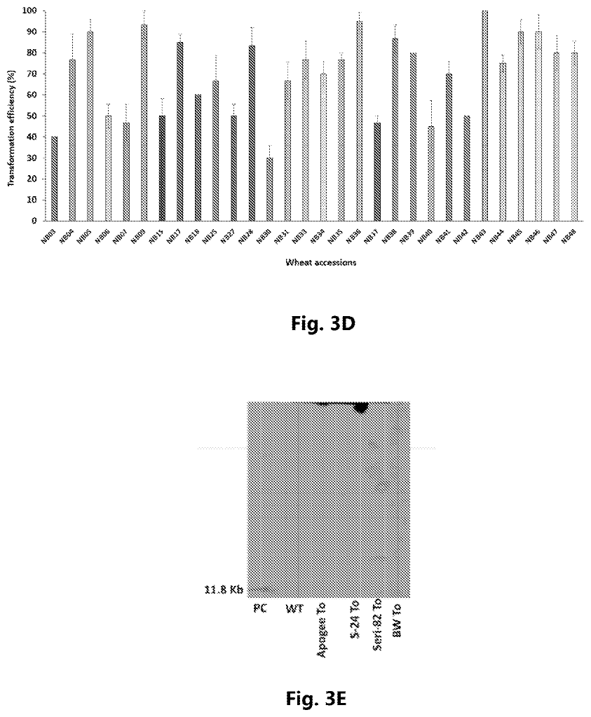

Seeds were collected from matured wheat spikes. The putative transgenic seeds (at T₀ generation) were plated on antibiotic plates (MS+200 mg/L hygromycin and 160 mg/L timentin; FIG. 3A) for resistance screening. Prior antibiotic resistance optimization trial showed that a hygromycin concentration of 200 mg/L is sufficient to arrest completely the germination and growth of wild-type plants in two genotypes ‘Bobwhite’ and ‘Yecora-Rojo’. Transformation efficiency was estimated based on the seeds that germinated on MS+200 hygromycin plates at 20° C. GUS staining (FIG. 3B) and PCR amplification (FIG. 3C) of the hygromycin (Hyg) resistance gene from leaves of transformed wheat were performed to validate those seeds that germinated under 200 mg/L hygromycin.

For GUS staining, two-weeks-old seedlings, and leaves tissue from plants at 24 days post-germination were used. Samples were incubated with phosphate buffer at pH 7.0 for 3 hours at 37° C. After incubation, the buffer was discarded, and samples were placed in X-GLU solution (X-glu, 5 mM potassium ferricyanide, 5 mM potassium ferrocyanide, 10 μl Triton X-100 in 0.1 M phosphate buffer, pH 7.0). Samples were then vacuum infiltrated for 15 minutes. The preparations were incubated at 37° C. for 48 hours. The blue color development was recorded at the end of the experiment.

For PCR assay, leaves were collected from antibiotic-resistant seedlings and wild-type control plants 24 days after germination. One square millimeter pieces of leaves were placed in 1.25% SDS solution and boiled at 95° C. for 20 minutes. One ml of supernatant was used as template for PCR reaction after cooling at room temperature. The PCR was performed using the primers specific to the Hyg resistance gene with forward and reverse primers following instructions from MyTaq Plant PCR kit (Meridian Life Science, Inc., US). PCR conditions were 95° C. for 30 seconds for denaturation, 51° C. for 45 seconds for annealing, and 72° C. for 1 minute for extension.

Integration of the transgene in T₀ transformation was confirmed using southern blot analysis, as shown in FIG. 3E. Southern blot analysis was performed by isolating genomic DNA from the T₀ transformants using the CTAB (cetyl trimethyl ammonium bromide) method. Fresh tissue, 4-5 g, was ground with a mortar and pestle, and then cooled with liquid nitrogen. Twenty ml (20 ml) of CTAB buffer (1 M Tris-HCl pH 8.0, 5 M NaCl, 0.5 M EDTA, 2% CTAB, and 1% (v/v) β-mercaptoethanol) was added, vortexed, and incubated at 65° C. for 30 min. After incubation, 20 ml of chloroform:isoamyl alcohol 24:1 (v/v) was added. The lysates were gently mixed and centrifuged at 4,000 g at 4° C. for 20 min. The upper aqueous phase was transferred to new tube, and total DNA was extracted and precipitated with 3 M sodium acetate at pH 5.2 in ice cold isopropanol. The precipitated DNA was dissolved in TE (Tris-Cl/EDTA) buffer. Twenty-five micrograms of extracted DNA was digested with SacI (Thermoscientific). Digested DNA was separated on 0.8% (w/v) agarose 1×TAE gel and electroblotted onto a positively charged nylon membrane (Thermoscientific) using 1×TAE as the transfer buffer. The DNA was cross-linked to the membrane using a microwave on a high setting (1800 watts) for 2 minutes. The DNA was then pre-hybridized at 65° C. for 15 min in Church buffer (sodium phosphate buffer containing 0.5 M Na₂HPO₄, pH 7.2, 20% (w/v) SDS, 1 mM EDTA, and 1% BSA) and hybridized overnight at 65° C. with 50 ng of DNA probe. The probes were prepared using biotin-11-dUTP (Thermoscientific). The probe for pCAMBIA1305.1 was amplified with Hyg resistance gene with forward with forward and reverse primers at a 53° C. annealing temperature. After hybridization, washing, blocking and detection was performed following manual of Chemiluminescent nucleic acid detection module.

The method was extended to different wheat varieties. Thirty spring growth habit genotypes of wheat including 27 Triticum aestivum accessions and 3 Triticum durum accessions were transformed. The transformation efficiencies, calculated by dividing the number of seeds grown in antibiotic selection media to the number of seeds placed in hygromycin (200 mg/L) media, for these 30 accessions ranged from about 30% to nearly 100%, as shown in FIG. 3D. Table 1 summarizes the transformation efficiencies across eight genotypes. Based on these results, the method appears to be genotype independent.

TABLE 1
Transformation efficiency in genotype-independent
manner on a group of hexaploidy and tetraploid
wheat representing many geographic regions.

| Accessions | Transformation efficiency | SEM |
|---|---|---|
| Chinese spring | 40.0 | 0.0 |
| Cadenza | 76.7 | 12.0 |
| Yecora-Rojo | 90.0 | 5.8 |
| Central Red | 50.0 | 5.8 |
| Glenlea | 46.7 | 8.8 |
| Ruby | 93.3 | 6.7 |
| Kronos | 50.0 | 8.2 |
| T-27 | 85.0 | 4.1 |
| T-28 | 60.0 | 0.0 |
| Gilat | 66.7 | 12.0 |
| Sonora64 | 50.0 | 5.8 |
| NacozariF76 | 83.3 | 8.8 |
| Veery19 | 30.0 | 5.8 |
| Cranbrook | 66.7 | 8.8 |
| Halbert | 76.7 | 8.8 |
| Apogee | 70.0 | 5.8 |
| Opata | 76.7 | 3.3 |
| Inia 66 | 95.0 | 4.1 |
| Seri-82 | 46.7 | 3.3 |
| S-24 | 86.7 | 6.7 |
| Fielder | 80.0 | 0.0 |
| Bobwhite | 45.0 | 12.2 |
| Line 28 | 70.0 | 5.8 |
| Line 43 | 50.0 | 0.0 |
| Line 10 | 100.0 | 0.0 |
| Verde | 75.0 | 4.1 |
| Ulen | 90.0 | 5.8 |
| Lassik | 90.0 | 8.2 |
| Patwin | 80.0 | 8.2 |
| Ke-Qun | 80.0 | 5.8 |

The method was also extended to different cereal crops. Barley (Rasmusson, Quest, and Robust), oat (INO9201 and Excel), sorghum (RTX423, and TX623), and rice were transformed using the method similar to wheat, except for barley and oat transformation the pCAMBIA1305.1 vector was used, for sorghum transformation the pCAMBIA1305.1 and PIP2-GUS-Bar vectors were used, and for rice the PIP2-GUS-Bar vector was used. The vectors were changed because the pCAMBIA1305.1 vector is a plant binary vector which has the Hyg resistance gene for selection of plants in hygromycin containing media and a β-glucuronidase gene driven using CaMV35s promoter, whereas the PIP2-Bar-GUS is plant binary vector has a bialaphos (Bar) resistance gene and a β-glucuronidase gene for selection of transformants using herbicide (basta) and GUS staining. Agrobacterium strain AGL1 was used for all transformation experiment. Plants transformed with pCAMBIA1305.1 were screened with GUS staining and plants transformed with PIP2-Bar-GUS vector were screened with leaf painting assay using 2% basta.

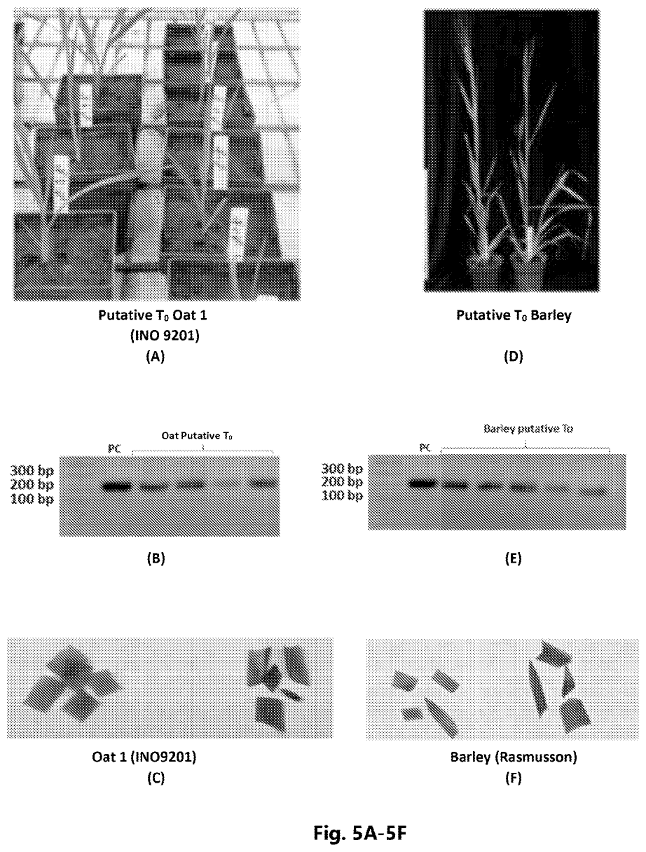

Two oat varieties (INO9201 and Excel) and three barley accessions (Rasmusson, Quest and Robust) were used for transformation. Transformation efficiency was calculated based on selection on hygromycin (200 mg/l) media, PCR amplification of the GUS gene, and GUS staining. The putative T₀ oat (INO9201) and barley were similar to wild type plants (FIG. 5A, 5D). The screening of positive transformants was confirmed using PCR amplification of GUS gene (FIG. 5B. 5E), and GUS staining of leaf samples of oat (INO9201) and barley (Rasmusson) (FIG. 5C, 5F). We obtained 47% transformation efficiency in oat (INO9201) accession and 12.7% transformation efficiency in barley (Rasmusson) accession.

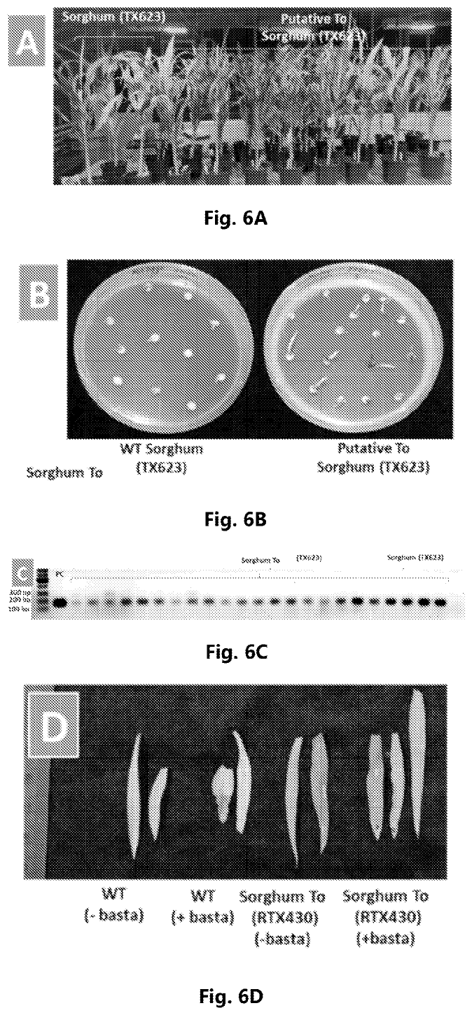

Sorghum transformants were phenotypically similar to wild type plants (FIG. 6A). Transformation efficiency of the sorghum putative T₀ transformants was calculated based on growth on an antibiotic selection media (hygromycin 200 mg/l and 160 mg/l timentin) (FIG. 6B), and using PCR amplification of the GUS gene (FIG. 6C). Basta resistance in sorghum putative T₀ transformants plants was screened by painting leaves with basta (2%) (FIG. 6D).

Based on these results, the method appears to be applicable across various cereals.

### Example 3

### Testing the Inheritance of Transgene in T₁ Generation in Wheat Genotypes.

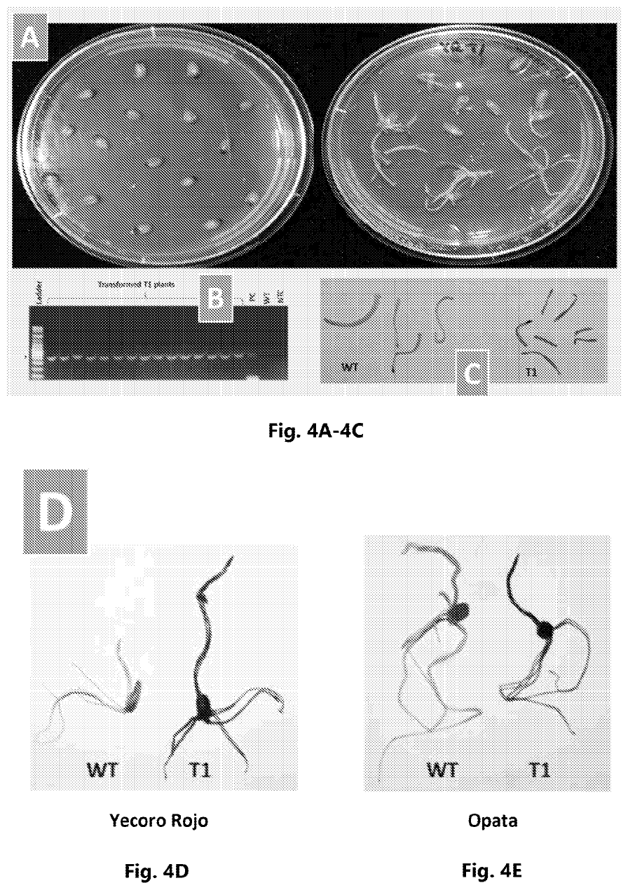

T₀ plants were grown to maturity under normal condition. Seeds from T₀ plants were harvested and plated as T₁ generation in antibiotic selection medium (200 mg/L hygromycin). The segregation ratio in T₁ generation was calculated as the ratio of the number of germinated seeds to the total number of seeds placed in an MS plate containing antibiotic selection medium. The ‘Yecora-Rojo’ putative T₁ plants were germinated in antibiotic selection media but germination of seeds with wild-type genotypes was inhibited in antibiotic selection media (FIG. 4A). The inheritance of the transgene was verified by PCR amplification of the Hyg resistance gene (FIG. 4B). Furthermore, GUS staining of two-weeks-old roots of T₁ seeds germinated in antibiotic selection was positive (FIG. 4C, 4D). T₁ seeds were germinated in antibiotic selection media (Table 2), and seedlings eventually showed GUS staining phenotype.

TABLE 2
Inheritance of transgene in T₁

| | No. seeds | No. seeds | Germination |
|---|---|---|---|
| Genotype | plated | germinated | percentage |
| Apogee | 32 | 25 | 78.12 |
| Yecora-rojo | 22 | 16 | 72.72 |
| Bobwhite | 33 | 26 | 78.78 |
| S-24 | 38 | 29 | 76.31 |

Methods may also comprise growing one or more successive generations of transgenic plants from transgenic seeds generated in accordance with methods herein.

The method described herein is an efficient, genotype-independent method of transforming cereals that is independent of tissue culture. The method effects a genetic transformation of a cereal in planta by taking a cereal plant that has at least one seed with a developing embryo and wounding the developing embryo post-anthesis while leaving the seed attached to the plant. The plant with the seed having the wounded developing embryo is co-incubated with a transgene, and the seed is allowed to fully mature while remaining attached to the plant. When the seed reaches physiological maturity, the seed is collected by removing the seed from the plant. The collected seed is screened for genetic transformation, and the transgenic seeds are isolated from the collected seeds.

The term “about” as used herein can allow for a degree of variability in a value or range, for example, within 10%, within 5%, or within 1% of a stated value or of a stated limit of a range.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation.

Any use of section headings and subheadings is solely for ease of reference and is not intended to limit any disclosure made in one section to that section only; rather, any disclosure made under one section heading or subheading is intended to constitute a disclosure under each and every other section heading or subheading.

Various modifications and variations of the described compositions, methods, and uses of the technology will be apparent to those skilled in the art without departing from the scope and spirit of the technology as described. Although the technology has been described in connection with specific exemplary embodiments, the invention as claimed should not be unduly limited to such specific embodiments. Indeed, various modifications of the described modes for carrying out the invention that are obvious to those skilled in the art are intended to be within the scope of the following claims.

The terms and expressions employed herein are used as terms of description and not of limitation. In this regard, where certain terms are defined and are described or discussed elsewhere, the definitions and all descriptions and discussions are intended to be attributed to such terms. There also is no intention in the use of such terms and expressions of excluding any equivalents of the features shown and described or portions thereof.

Further, all publications and patents mentioned herein are incorporated by reference in their entireties for all purposes. In the event of inconsistent usages between this document and those documents so incorporated by reference, the usage in the incorporated reference should be considered supplementary to that of this document; for irreconcilable inconsistencies, the usage in this document controls.

What is claimed is:

1. A method of transforming a wheat or other cereal in planta, the method comprising:
(i) co-incubating, post-anthesis, a cereal inflorescence of developing embryos with an inoculation medium, wherein the inoculation medium comprises a transgene-modified Agrobacterium;
(ii) collecting, upon the cereal inflorescence reaching maturity, seeds produced from at least a portion of the developing embryos; and
(iii) selecting one or more transgenic seeds from among the collected seeds.

2. The method of claim 1 wherein the cereal is selected from Triticum (wheat), Sorghum (sorghum), Oryza (rice), Avena (oats), Zea (corn), Hordeum (barley), and Secale (rye).

3. The method of claim 1, further comprising exposing at least a portion of the developing embryos prior to co-incubation, wherein exposing comprises trimming awns, removing outer glumes, and plucking out middle florets of each respective embryo.

4. The method of claim 3, further comprising wounding the exposed, developing embryos prior to co-incubation.

5. The method of claim 4, wherein the exposed, developing embryos are wounded at about 7 days to about 24 days after anthesis.

6. The method of any one of claims 1-5, wherein co-incubating comprises immersing the cereal inflorescence in the inoculation medium.

7. The method of claim 6, wherein the cereal inflorescence is immersed in the inoculation medium immediately after the exposed, developing embryos are wounded.

8. The method of claim 7, wherein the cereal inflorescence is co-incubated with the inoculation medium for about 10 hours to about 72 hours and then covered until maturity.

9. The method of claim 8, wherein, after co-incubation, the cereal inflorescence is covered with a paper bag.

10. The method of claim 8, wherein the cereal inflorescence is exposed to light at about 90 to about 1,500 μmolm⁻²s⁻¹ for up to about 16 hours daily.

11. The method of any one of claims 1-5, wherein the cereal inflorescence is co-incubated with the inoculation medium at a temperature of about 25±5° C.

12. The method of any one of claims 1-5, wherein the inoculation medium is shaken gently once every 6 hours to about every 12 hours.

13. The method of any one of claims 1-5, wherein the cereal inflorescence is co-incubated with the inoculation medium under vacuum infiltration.

14. The method of claim 13, wherein vacuum infiltration is at about 55 PSI.

15. The method of claim 14, wherein the vacuum infiltration lasts from about 10 minutes to about 60 minutes.

16. The method of claim 15, wherein the vacuum infiltration lasts about 30 minutes.

17. The method of claim 15, wherein, after vacuum infiltration, the cereal inflorescence is maintained at a temperature of about 20±5° C.

18. The method of any one of claims 1-5, wherein the cereal inflorescence is exposed to light at about 90 to about 1,500 μmolm⁻²s⁻¹ for up to about 16 hours daily.

19. The method of any one of claims 1-5, wherein the Agrobacterium is a strain selected from the group consisting of AGL1, EHA105, GV1301, At503, LBA4404, EHA101, and C58C1.

20. The method of any one of claim 19, wherein the Agrobacterium is the strain AGL1.

21. The method of any one of claims 1-5, wherein the transgene is in a vector selected from pCAMBIA1305.1, PIP2-GUS-Bar, or a combination thereof.

22. The method of any one of claims 2-5, wherein the cereal is a Triticum (wheat) variety.

23. The method of claim 22 wherein the Triticum (wheat) variety is selected from AG0762, Apogee, Bobwhite, Cadenza, Canvas, Central Red, Chinese spring, Cranbrook, Fielder, Gallagher, Gilat, Glenlea, Halbert, IN0316, Inia 66, Ke-Qun, Kronos, Lassik, Line 10, Line 28, Line 43, NacozariF76, Opata, Patwin, Red Fife, Ruby, S-24, Seri-82, Sky dance, Smith's Gold, Sonora64, T-13, T-27, T-28, T-38, Ulen, Veery 19, Verde, and Yecora-Rojo.

24. The method of any one of claims 2-5, wherein the cereal is a Hordeum (barley) variety.

25. The method claim 24, wherein the Hordeum (barley) variety is selected from Rasmusson, Quest, and Robust.

26. The method of any one of claims 2-5, wherein the cereal plant is an Avena (oats) variety.

27. The method of claim 26, wherein the Avena (oats) variety is selected from INO9201 and Excel.

28. The method of any one of claims 2-5, wherein the cereal plant is a Sorghum (sorghum) variety.

29. The method claim 28, wherein the Sorghum (sorghum) variety is selected from RTX423 and TX623.

30. The method of any one of claims 1-5, further comprising:
(iv) growing a plant from each of one or more of the transgenic seeds of (iii);
(v) collecting seeds from the plants of (iv); and
(vi) selecting for transgenic seeds among the collected seeds of (v).

31. A transgenic plant obtained in accordance with method of claim 30.

32. A cell, tissue, or organ obtained from the transgenic plant of claim 31.

33. A transgenic seed obtained from the transgenic plant of claim 31.

34. A T2 plant obtained from the transgenic seed of claim 33.
